SEQUENCE LISTING (5 sequences)

SEQ ID NO: 1 - protein, 503 aa, Homo sapiens
MEPAGPAPGRLGPLLCLLLAASCAWSGVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 2 - protein, 288 aa, Homo sapiens [sequence omitted]

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 15 aa, Artificial Sequence
  It is synthesized.
GGGGSGGGGSGGGGS

SEQ ID NO: 5 - protein, 499 aa, Artificial Sequence
  It is translated by engineering cDNA.
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTER

(19) United States
(12) Patent Application Publication
(10) Pub. No.: US 2020/0087377 A1
(43) Pub. Date: Mar. 19, 2020

(54) Multifunctional Fusion Protein and Applications Thereof

(71) Applicant: JIANGSU CTL BIOLOGICAL TECHNOLOGY CO., LTD, Taizhou (CN)

(72) Inventors: XiLian YUE, Taizhou (CN); WeiXing SHI, Taizhou (CN); ChunXia YANG, Taizhou (CN); Ying CHEN, Taizhou (CN); Tao TANG, Taizhou (CN)

(21) Appl. No.: 15/747,128

(22) PCT Filed: Dec. 11, 2017
(86) PCT No.: PCT/CN2017/115458

§ 371 Date: Jan. 23, 2018

(30) Foreign Application Priority Data
Jul. 3, 2017 (CN) 201710531457.9
Oct. 23, 2017 (CN) 201711016798.9

(51) Int. Cl. C07K 14/705

(52) U.S. Cl. CPC C07K 14/70521; A61K 38/00; C07K 2319/30; C07K 14/70596

(57) ABSTRACT

A multifunctional fusion protein and applications thereof. The multifunctional fusion protein comprising a. functional domains that recognize CD47 positive tumor cells: the extracellular portion of SIRPα, b. functional domains that recognize PD-L1-positive tumor cells: the extracellular portion of PD-1, and c. functional domains that bind to immune cells: high-affinity human IgG1Fc portion. The fusion protein of the invention can meet the needs of the patients for tumor immunotherapy. The recombinant fusion protein can recognize CD47 and PD-L1-positive tumor cells, and bind with immune effector cells containing Fc receptors.

This application is the U.S. national phase of International Application No. PCT/CN2017/115458 filed on 11 Dec. 2017 which designated the U.S. and claims priority to Chinese Application Nos. CN201710531457.9 & CN201711016798.9 filed on 3 Jul. 2017 & 26 Oct. 2017, respectively, the entire contents of each of which are hereby incorporated by reference.

### TECHNICAL FIELD

The present invention relates to a fusion protein technical field, particularly relates to a multifunctional fusion protein and application thereof in the treatment of cancers.

### BACKGROUND ART

Cancer is a common most serious disease that threatens people's life and quality of life. At present, clinically used drugs for the treatment of cancers have shortcomings. For example, chemotherapy drugs have great side effects, targeted drugs are prone to drug resistance (Curr Pharm Des. 2010, 16:3-10), immune checkpoint inhibitors alone have low clinical efficacy (NEM 2012, 366:2443-2454), and chimeric antigen receptor T (Car-T) cell therapy have cytokine storm and high recurrence rate (Curr Opin Pediatr. 2017, 29:27-33). Therefore, it is the most urgent task and project to find novel efficient, low-toxicity drugs for the treatment of cancers to reduce the mortality and improve the quality of life of patients in the medical and health care field in China and across the world.

The occurrence of tumor is caused by gene mutations in the cell division process and loss of regulatory control of the growth of muted cells. Because tumor cells evade immune surveillance by producing factors that suppress immune responses, the inhibitors secreted by tumor cells can cause immune cell inactivation and can not kill or clear tumor cells, even though there are immune cells within the tumor and in the surrounding environment of the tumor (J Immunol 2005; 175: 6169-6176). Only by blocking the tumor immunosuppressive factors, activating immune cell populations and allowing the body's immune system to regain the function of killing antigen-positive target cells, can the tumor be cleared (Trends Immunol. 2015; 36:265-276) and ultimately to cure cancers.

CD47 is a multifunctional protein that, in conjunction with its ligands, can produce a series of functions such as cell growth, migration and autoimmunity (Nat Med 2015; 21:1122-3), and induce inhibitory signals by binding with signal regulatory protein-a (SIRPα) expressing in macrophages and antigen presenting cell surfaces, to prevent macrophage endocytosis of CD47-positive cells (Trends Cell Biol 2001, 11:130-135; Science 2000, 288:2051-2054). The extensive expression of CD47 on erythrocytes, platelets, lymphocytes and stem cells in peripheral blood is also a major mechanism by which these cells escape macrophage phagocytosis (J Exp Med 2001, 193:855-862.; Leuk Lymphoma 2004, 45:1319-1327; Cell 2009, 138:271-285). One of the ways that tumors produce immunosuppression is to suppress the body's immune response to the tumor through its CD47 and its ligand signaling pathway. More and more evidences have shown that, CD47 is universally expressed on the surface of various solid tumor cells (PNAS 2012, 109: 6662-6667), and the solid tumors highly expressing CD47 can evade macrophage recognition and endocytosis, which is one of the mechanisms by which the tumors evade immune surveillance for growth and spreading (Trends Immunol 2010, 31:212-219). In addition, the expression level of CD47 in tumor cells is significantly negatively correlated with patient's survival time (Trends Cell Biol 2001, 11: 130-135). The existing evidences have demonstrated that, blocking the interaction of CD47 with SIRP with antibodies can increase the endocytosis of tumor cells by macrophages and inhibit the spread and metastasis of tumors (J Clin Invest 2016; 126: 2610-20; PLoS ONE 10 (9): e0137345). However, due to the high affinity of antibodies, excessive immunosuppression can result in the lack of erythrocytes that express CD47, leading to extreme anemia (eLife 2017; 6: e18173).

Another way for tumors to produce immunosuppression is to suppress the body's immune response to the tumors through PD-1 and its ligand signaling pathways. The PD-1 receptor (CD279) is generally expressed on the surface of activated or depleted T cells and its ligand PD-L1 (B7-H1; CD274) is generally expressed on the surface of tumor cells. PD-L1 positive tumors cells can promote tumor cells to express stem cell signals, prompting tumors to spread and metastasize more easily (Signal Transduction and Targeted Therapy 2016, 1: 16030). In addition, the over-expressive PD-L1 on tumor cells binds with T-cell surface PD-1, to convey inhibitory signals, reduce the secretion of T-cell killer cytokines, inhibit T cell function and create a tumor immunosuppressive microenvironment so that T Cells lose the ability to kill target cells, eventually resulting in tumor cell escape (Clin Cancer Res. 2012, 18: 6580). Since tumor-infiltrating immune cells overexpress PD-1 (Blood. 2009, 114: 1537), tumor cells expressing PD-L1 are more likely to inactivate tumor-infiltrating immune cells and lose the ability to kill tumor cells (Curr Opin Immunol. 2012, 24: 207). The high expressions of PD-L1 in tumor cells have been widely validated in various tumors and its expression level is significantly negatively correlated with the patient's clinical survival (PLoS One. 2011, 6: e17621). Antibodies can specifically recognize protein antigens on the surface of target cells. Checkpoint inhibitory antibodies can block immunosuppressive signals on the surface of depleted cells, for example, by binding of PD-1 antibodies Pembrolizumab and Nivolumab to PD-1, it can prevent conduction and activation of inhibitory signals and recover the functions of depleting immune cells, showing an unprecedented clinical effect in the treatment of cancer, especially with complete disappearance of tumor in some patients (NEM 2012, 366: 2455-2465; NEM 2012, 366: 2443-2454). However, the current clinical practice of blocking signal transduction alone is unsatisfactory (NATURE 2014, 515: 568-571) and the T cells have a short duration of recovery (Science 2016, 354: 1160-1165), the in vivo specific immune cells of the patient will soon return to failure state (Science 2016, 354: 1165-1169), limiting its clinical therapeutic effect.

Humoral responses to antigens are one of the major immune responses to tumors in the body. It has been decades of years for the treatment of tumors with monoclonal antibodies (Cell 2012; 148:1081-4). The mechanism of tumor control is gradually becoming clear (Cell 2012; 148:1081-4), including direct binding of antigens on target cells, hindering the binding of receptors to the cell growth factor, causing apoptosis in target cells (Clin Oncol 2009; 27:1122-9); causing indirect complement-dependent cytotoxicity (CDC) by the binding of Fc on the antibody to C1q; causing antibody-dependent cell phagocytosis (ADCP) by the recognition and binding of antibody Fc to NK cells (J Hematol Oncol 2013; 6:1; Cancer Res 2011; 71:5134); and producing antibody-dependent cell phagocytosis (ADCP) through the binding of antibodies to macrophages. However, ADCP effects are inhibited by tumor cells expressing CD47 and PD-L1 (PNAS 2011; 108: 18342-7; Nature 2017, doi: 10.1038/nature22396). Therefore, the effect of antibodies alone can only achieve limited clinical effects (PNAS 2011, 108: 18347).

Up to now, no protein drugs that can block various immunosuppressive signal pathways are available clinically, while the drugs targeting on a single pathway have the disadvantage of low curative effect.

### SUMMARY OF THE INVENTION

The object of the present invention is to overcome the drawbacks of the prior art and provide a fusion protein that blocks immune cell inhibitory signaling pathways. It can block both the CD47 and SIRPα inhibitory signaling pathway, and PD-1 and PD-L1 inhibitory signaling pathway; in addition, it can increase the ability of immune cells that are produced by the antibody Fc signals to kill the target cells of tumors.

To achieve the above object, the present invention employs the following technical solutions.

The present invention provides a multifunctional fusion protein capable of recognizing tumor cells and binding to immune cells, comprising:

a. Functional domains that recognize CD47 positive tumor cells: the extracellular portion of SIRPα,

b. Functional domains that recognize PD-L1-positive tumor cells: the extracellular portion of PD-1,

c. Functional domains that bind to immune cells: high-affinity human IgG1Fc portion,

d. Non-functional amino acid fragments that bind the above functional domains, making no mutual interference of the protein folding of each functional domain. It can bind to CD47 and PD-L1 on the tumor, and also bind to NK cells with Fc receptors and macrophages, to promote killing and clearance of tumor tissues by immune cells, playing the multi-functional features of fusion proteins.

The surface receptors of tumor cells include PD-L1 and CD47, having the functions of immune checkpoint of co-suppressive immune and transmitting immunosuppressive signals. The overexpression of such receptor genes on the surface of tumor cells causes tumor cells to evade the killing and clearance of immune cells. The fusion protein SIRPα fragment partially blocks the binding of SIRPα to CD47and signal transduction in immune cells, and the fusion protein PD-1 fragment partially blocks the immunosuppression caused by the binding of PD-1 in immune cells to PD-L1 expressed by tumors, to prevent the inhibitory effect of the checkpoint ligand in the tumor microenvironment on the immune system. The recognition functional domains of fusion protein can recognize the surface receptors of tumor cells using specific ligands.

In order to further optimize the above technical solutions, the present invention further adopts the following technical measures:

The extracellular portion of SIRPα is the sites 31-150 in the amino acid sequence shown in SEQ ID NO: 1 or a mutant containing at least 90% of the same sequence as the above sites.

The extracellular portion of PD-1 is the sites 26-147 in the amino acid sequence shown in SEQ ID NO: 2 or a mutant containing at least 90% of the same sequence as the above sites.

The high-affinity human IgG1Fc portion is the sites 1-227 in the amino acid sequence shown in SEQ ID NO: 3 or a mutant containing at least 90% of the same sequence as the above sites. The human antibody IgG1Fc binds to immune cell surface Fc receptor in the fusion protein, and brings the functional immune cells close to the target cells, so that killer cells are prone to produce killing effect on the target cells, to prevent off-target effect easily caused by single-target binding.

The non-functional amino acid fragment is the amino acid sequence shown in SEQ ID NO: 4 or a mutant containing at least 90% of the same sequence as the above site.

The complete amino acid sequence of the multifunctional fusion protein is as shown in SEQ ID NO: 5 or a mutant containing at least 90% of the same sequence as the above site.

The multifunctional fusion protein of SEQ ID NO: 5 is prepared according to the following steps:

Step 1): Connect the extracellular fragments of human SIRPα, extracellular fragments of PD-1 and corresponding amino acid sequence bases of IgG1Fc by the non-functional amino acid bases through gene synthesis, to form structural genes of fusion protein;

Step 2): Transform the structural genes to a mammalian expression vector and then transfect into hamster ovary cells;

Step 3): Incubate the hamster ovary cells in an incubator for a period of time, take the supernatant and purify to obtain a recombinant fusion protein.

The above multifunctional fusion protein can be used in the preparation of a medicament for the treatment of tumors expressing CD47 and PD-L1.

The above fusion protein can be used alone or in combination with chemotherapy, targeted drugs, antibody drugs and cell therapy for the preparation of a medicament for the treatment of tumors expressing CD47 and PD-L1. The tumor diseases include solid tumors and hematological cancers. The cancers include renal cell carcinoma, melanoma, lymphoma, colorectal cancer, liver cancer, ovarian cancer, head and neck squamous cell carcinoma, bladder cancer, lung cancer and leukemia.

The fusion protein of the invention can meet the needs of the patients for tumor immunotherapy. The recombinant fusion protein can recognize CD47 and PD-L1-positive tumor cells, and bind with immune effector cells containing Fc receptors. When used clinically, it can enhance the functions of inhibiting tumor growth and controlling virus infection, showing good clinical prospects and extensive applications.

The fusion proteins designed and produced according to the present invention can overcome the shortcomings of the existing drugs, block the multiple immunosuppressive signaling pathways and increase the killing of antibody-dependent immune cells on the tumor, thereby improving the inhibition and elimination of tumor by the immune system and enhancing the clinical efficacy.

### BRIEF DESCRIPTION OF THE DRAWINGS

FIG. 1 shows a gel electrophoresis analysis chart of a recombinant fusion protein prepared according to an embodiment of the present invention.

FIG. 2 shows an anti-cancer test of ascites of a recombinant fusion protein in patients with colorectal cancer according to an embodiment of the present invention.

FIG. 3 shows an in vitro test of a recombinant fusion protein on promoting macrophage phagocytosis of tumor cells according to an embodiment of the present invention.

FIG. 4 shows an in vitro phagocytic index of macrophages on different tumor cells by recombinant fusion proteins prepared according to an embodiment of the present invention.

### DETAILED DESCRIPTIONS OF THE PREFERRED EMBODIMENTS

The present invention provides a multifunctional fusion protein comprising two functional domains that specifically recognize tumor cells and a functional domain that binds to Fc receptors of immune cells. The functional domains are connected by non-functional amino acid fragments with a certain length, and produced and purified by the way of expressions in mammalian cells. The present invention further provides the applications of the fusion proteins in the treatment of cancers.

The present invention is further described below with reference to the accompanying drawings and embodiments. The following embodiments are only used for more clearly illustrating the technical solutions of the present invention, and are not intended to limit the protection scope of the present invention.

In the embodiments, tumor cells that express CD47 and PD-L1 refer to solid tumor or hematological tumor cells. Immune cells that express Fc receptors refer to NK cells or macrophages or monocytes.

The complete amino acid sequence of SIRPα is shown as SEQ ID NO: 1 (GenBank: BC038510.2), and the complete amino acid sequence of PD-1 is shown as SEQ ID NO: 2 (GenBank: L27440.1). The complete amino acid sequence of human antibody IgG1Fc in the fusion protein is based on GenBank: AAC82527.1, including the sequence shown in SEQ ID NO: 3. The amino acid sequence that connects to fusion protein functional domain comprises the sequence shown in SEQ ID NO: 4.

The amino acid sequence of complete fusion protein consisting of the extracellular functional domain of SEQ ID NO: 1 (AA31-150), the extracellular domain of SEQ ID NO: 2 (AA26-147), SEQ ID NO: 3 (AA103-329), and SEQ ID NO: 4 is the sequence shown in SEQ ID NO: 5.

### Embodiment 1

This embodiment described the gene construction and purification of a recombinant fusion protein.

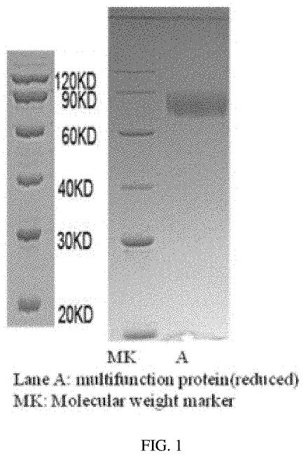

According to the bases of functional domains of human SIRPα, IgG1Fc and PD-1, multifunctional fusion protein (SEQ ID NO: 5) genes were made by gene synthesis and ligated with bases of non-functional amino acid fragments, then transferred into eukaryotic expression vector pcDNA3.1. The multifunctional fusion protein gene was transferred into the eukaryotic expression vector by gene digestion and cloning. Finally fusion protein vector was transfected into Chinese hamster ovary cells (CHO). Transfected cells were cultured in a 37° C., 5% CO2 incubator, 72 hours later, the supernatant was fetched and further purified by ProteinA affinity chromatography, finally the purified protein was a multi-functional recombinant fusion protein. The molecular weight of purified protein was confirmed by electrophoresis, demonstrating that the recombinant fusion protein designed according to the present invention can be produced by CHO cells. Finally, the concentration of the protein was measured with a spectrophotometer and diluted and saved in PBS, for further in vitro and in vivo activity testing and functional studies. In FIG. 1, the gel electrophoresis test showed that the molecular weight of fusion protein conformed to the design expectation.

### Embodiment 2

This embodiment described the tumor cell killing test of a multi-functional recombinant fusion protein in the ascites of patients with colorectal cancer.

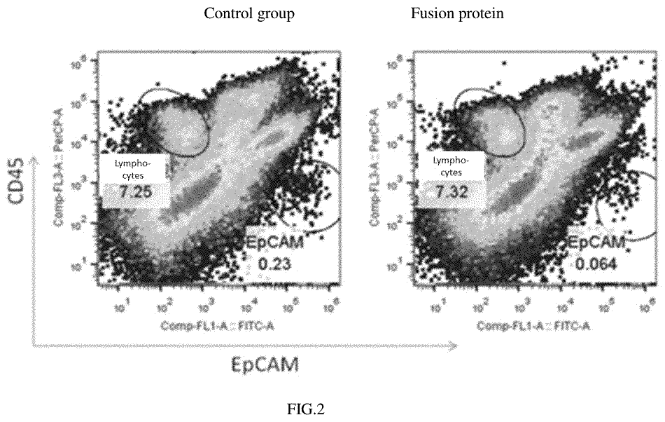

Ascites from patients with colorectal cancer containing tumor cells and immune cells were divided into two groups and placed in a 24-well plate at 1 ml per well. In the control group, PBS was added, and in the experimental group, fusion protein was added to a final concentration of 1 μg/ml. After mixing evenly, it was incubated at 37° C. for 48 hours in a 5% CO2 incubator. Cells were collected and washed once with PBS and then stained with flow cytometry for immune cells (CD45) and tumor cells (EpCAM) for 20 minutes, after washed, determined by flow cytometry. FIG. 2 showed that, compared to ascites in patients without fusion proteins, the fusion protein treatment significantly reduced the proportion of tumor cells in ascites (0.23% vs 0.064%), but without changing the proportion of lymphocytes (7.25% vs. 32%), which demonstrated that fusion proteins could selectively kill tumor cells and have the application values for the treatment of cancers.

### Embodiment 3

This embodiment described in vitro test of a multi-functional recombinant fusion protein on promoting macrophage phagocytosis of tumor cells.

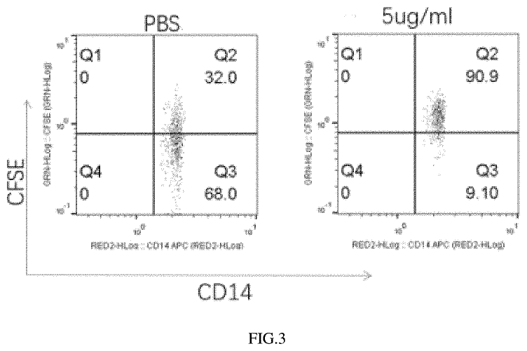

Transfer 1×10⁵ macrophages diluted in 100 ul DMEM medium to a 96-well plate, place to an incubator at 37 [symbol] , 5°% CO2 for 2 hours, then add 2×10⁵ tumor cells labeled with CFSE in 100 ul DMEM medium. Add fusion protein to a final concentration of 5 ug/ml as a fusion protein group, and those without fusion protein as a control group. After 2 hours of incubation, harvest all cells, centrifuge, add anti-CD14-depleted antibody for staining 20 minutes. After washing, conduct analysis by flow cytometry. FIG. 3 showed that the addition of the fusion protein promoted the phagocytosis of H358 tumor cells, as compared with the control group, the percentage of CFSE-containing CD14 cells increased from 32% in the control group to 90.9% in the fusion protein group.

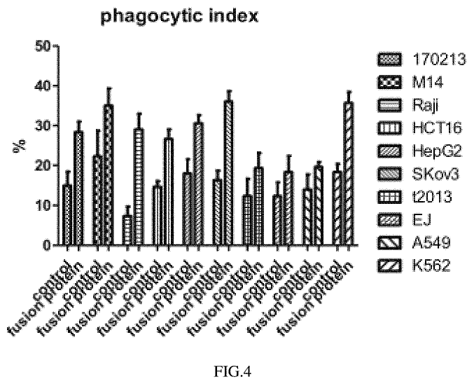

The in vitro phagocytosis of macrophages on different tumor cells was tested by above method. These cancers include renal cell carcinoma (170213), melanoma (M14), lymphoma (Raji), colorectal carcinoma (HCT116), liver cancer (HepG2), ovary cancer (SKOV3), head and neck squamous cell carcinoma (t2013), bladder cancer (EJ), lung cancer (A549) and leukemia (K562). The phagocytic efficiency was expressed by the phagocytic index and was calculated as: phagocytic index=100%×CFSE-positive CD14 cells/CD14 positive cells. The test results of all tumor cells were shown in FIG. 4. The fusion protein could promote phagocytosis of these tumor cells.

This embodiment showed the applications of a multi-functional recombinant fusion protein in the treatment of cancers caused by tumor immunosuppression. The above multi-functional recombinant fusion protein can be used alone or in combination with chemotherapy, targeted drugs, antibody drugs and cell therapy.

As can be seen from the above embodiments, the fusion protein capable of restoring the function of depleted immune cells according to the present invention can not only recognize antigen-positive tumor cells but also bind to immune cells to increase the function of immune cell-killing antigen-positive cells. Since tumorigenesis and proliferation are the result of immune escape from tumor cells expressing CD47 and PD-L1, fusion protein can both block the tumor's immunosuppressive pathway and promote the killing of immune cells on tumors. Therefore, the clinical application of the above fusion protein can strengthen the inhibition of tumor growth, having a good clinical prospect and a wide range of applications.

Specific embodiments of the present invention have been described above in detail, but these are merely examples, and the present invention is not limited to the specific embodiments described above. For those skilled in the art, any equivalent modifications and substitutions thereof fall within the scope of the present invention. Therefore, all the equivalent changes and modifications made without departing from the spirit and scope of the present invention shall fall within the scope of the present invention.

1. A multifunctional fusion protein capable of recognizing tumor cells and binding to immune cells, comprising:
an extracellular portion of SIRPα that recognizes CD47 positive tumor cells,
an extracellular portion of PD-1 that recognizes PD-L1-positive tumor cells,
a high-affinity human IgG1Fc portion that binds to immune cells,
non-functional amino acid fragment that binds the extracellular portion of SIRPα, the extracellular portion of PD-1 and the high-affinity human IgG1Fc portion; the non-functional amino acid fragment doesn't interfere protein folding of the extracellular portion of SIRPα, the extracellular portion of PD-1 and the high-affinity human IgG1Fc portion while binds to the CD47 positive and the PD-L1 positive tumor cells, NK cells with Fc receptors and macrophages.

2. The multifunctional fusion protein according to claim 1, wherein the extracellular portion of SIRPα has an amino acid sequence shown in SEQ ID NO: 1 or at least 90% identical with the amino acid sequence shown in SEQ ID NO: 1.

3. The multifunctional fusion protein according to claim 1, wherein the extracellular portion of PD 1 has an amino acid sequence shown in SEQ ID NO: 2 or at least 90% identical with amino acid sequence shown in SEQ ID NO: 2.

4. The multifunctional fusion protein according to claim 1, wherein the high-affinity human IgG1Fc portion has an amino acid sequence shown in SEQ ID NO: 3 or at least 90% identical with the amino acid sequence shown in SEQ ID NO: 3.

5. The multifunctional fusion protein according to claim 1, wherein the non-functional amino acid fragment has an amino acid sequence shown in SEQ ID NO: 4 or at least 90% identical with the amino acid sequence shown in SEQ ID NO: 4.

6. The multifunctional fusion protein according to claim 1, wherein a complete amino acid sequence of the multifunctional fusion protein has an amino acid sequence shown in SEQ ID NO: 5 or at least 90% identical with the an amino acid sequence shown in SEQ ID NO: 5.

7. The multifunctional fusion protein according to claim 6, wherein the multifunctional fusion protein is prepared by the following steps:
step 1): connect the extracellular fragments of human SIRPα, extracellular fragments of PD-1 and corresponding amino acid sequence bases of IgG1Fc by the non-functional amino acid bases through gene synthesis, to form structural genes of fusion protein;
step 2): transform the structural genes to a mammalian expression vector and then transfect into hamster ovary cells;
step 3): incubate the hamster ovary cells in an incubator for a period of time, take the supernatant and purify to obtain a recombinant fusion protein.

8. (canceled)

9. A method for treating a tumor comprising a step of administering a multifunctional fusion protein of claim 1 to a subject in need of treating tumor treatment, wherein the tumor expresses CD47 and PD-L1.
